(19) United States
(12) Patent Application Publication
(10) Pub. No.: US 2010/0261163 A1
(43) Pub. Date: Oct. 14, 2010

(54) Method for simultaneous detection of Mycobacterium tuberculosis complex and identification of mutations in mycobacterial DNA resulting in the resistance of microorganisms to rifampicin and isoniazid on biological microarrays, set of primers, biochip, and set of oligonucleotide probes used in the method

(75) Inventors: Alexandr Sergeevich Zasedatelev, Moscow (RU); Alexander Yurievich Sobolev, Moscow (RU); Dmitry Alexandrovich Gryadunov, Moscow (RU); Sergei Anatolievich Lapa, Konakovo (RU); Vladimir Mikhailovich Mikhailovich, Moscow (RU); Andrei Darievich Mirzabekov, Moscow (RU); Natalia Vladimirovna Mirzabekova, Moscow (RU)

Correspondence Address: LADAS & PARRY LLP, 26 WEST 61ST STREET, NEW YORK, NY, 10023, US

(73) Assignee: INSTITUT MOLEKULYARNOI BIOLOGII IMENI V.A. ENGELGARDTA ROSSIISKOI AKADEMII NAUK

(21) Appl. No.: 11/645,841

(22) Filed: Dec. 26, 2006

(30) Foreign Application Priority Data
Dec. 26, 2005 (RU) 2005 140679

(51) Int. Cl. C12Q 1/68; C12M 1/34

(52) U.S. Cl. 435/6; 435/287.2

(57) ABSTRACT

The present invention relates to molecular biology, microbiology, and medicine and provides the method for detection of Mycobacterium tuberculosis complex with simultaneous evaluation of sensitivity of the strains to rifampicin and isoniazid in clinical sample on differentiating biochip. The method is based on two-stage multiplex PCR to obtain fluorescent DNA fragments followed by hybridization of these fragments on microarray containing the set of specific discriminating oligonucleotides. The determination of the resistance of Mycobacterium tuberculosis to rifampicin and isoniazid is carried out by evaluation of point nucleotide substitutions in DNA of microorganism. The present invention allows conduct analysis directly in clinical sample, to evaluate a number of mutations simultaneously, to decrease the cost price of analysis, and to reduce the time of its conducting. The present invention also relates to set of primers, biochip, and set of oligonucleotide probes used in realization of the method.

### FIELD OF THE INVENTION

The present invention relates to molecular biology, microbiology, and medicine and provides the method for detection of Mycobacterium tuberculosis complex (Mycobacterium tuberculosis, M. bovis, M. bovis BCG, M. africanum, and M. microti) with simultaneous evaluation of sensitivity of the strains to rifampicin and isoniazid in clinical sample on differentiating biochip.

### BACKGROUND OF THE INVENTION

The following methods are currently used for detection of the mutations responsible for drug-resistance of:

### I. Single Nucleotide Polymorphism (SNP);

Dubiley S., Kirillov E and A. Mirzabekov. 1999. Polymorphism analysis and gene detection by minisequencing on an array of gel-immobilized primers. Nucleic Acids Research, Vol. 27, No. 18 (e19).
Marth G T, Korf I, Yandell M D et al. 1999. A general approach to single-nucleotide polymorphism discovery. Nat Genet., December; 23 (4): 452-456.

### II. Allele Specific PCR;

De los Monteras L. E. E., J. C. Galan, M. Gutierrez, S. Samper, J. F. G. Marin, C. Martin, L. Doninguez, L. de Rafael, F. Baquero, E. Gomez-Mampaso, and J. Blazquez. 1998. Allele-Specific PCR Method Based on pncA and oxyR Sequences for Distinguishing Mycobacterium bovis from Mycobacterium tuberculosis: Intraspecific M. bovis pncA Sequence Polymorphism. J. Clin. Microbiol. 36: 239-242.

### III. Restriction Fragment Length Polymorphism (RFLP);

PCR-RFLP Detection of point Mutations in the Catalase-Peroxidase Gene (katG) of Mycobacterium tuberculosis Associated with Isoniazid Resistance. In: PCR Protocols for Emerging Infectious Diseases (D. H. Persing, Ed.) (1996) ASM Press, Washington, pp. 144-149.

### IV. Single- and (Double)-Strand Conformation Polymorphism (SSCP and DSCP);

Delgado M. B. and A. Telenti. Detection of Fluoroquinolone Resistance Mutations in Mycobacterium tuberculosis. In: PCR Protocols for Emerging Infectious Diseases (D. H. Persing, Ed.) (1996) ASM Press, Washington, pp. 138-143.
Pretorius G. S., P. D. van Helden, F. Sirgel, K. D. Eisenach and T. C. Victor. 1995. Mutations in katG Gene Sequences in Isoniazid-Resistant Clinical Isolates of Mycobacterium tuberculosis Are Rare. Antimicrob. Agents Chemother., 39, 10, 2276-2281.

### V. Hybridization on High-Density Microarrays;

Troesch A, Nguyen H, Miyada C G et al. Mycobacterium species identification and rifampin resistance testing with high-density DNA probe arrays. J Clin Microbiol 1999; 37: 49-55.
W. Sougakoff, M. Rodrigue, C. Truffot-Pernot, M. Renard, N. Durin, M. Szpytma, R. Vachon, A. Troesch and V. Jarlier. Use of a high-density DNA probe array for detecting mutations involved in rifampicin resistance in Mycobacterium tuberculosis. Clin Microbiol Infect. 2004; 10(4):289-94.

### VI. Hybridization on Specialized Oligonucleotide Microarrays;

Jun Yue, Wei Shi, Jingping Xie, Yao Li, Erliang Zeng and Honghai Wang. Detection of rifampin-resistant Mycobacterium tuberculosis strains by using a specialized oligonucleotide microarray. Diagn Microbiol Infect Dis 2004; 48: 47-54.
Gryadunov D. A., Mikhailovich V. M., Lapa S. A., Roudinskii N. I., Barskii V. E., Chudinov A. V., Zasedatelev A. S., Mirzabekov A. D. Identification of Mycobacterium tuberculosis strains with simultaneous evaluation of their drug-resistance by hybridization method on oligonucleotide microarrays. Molekulyarnaya genetika, mikrobiologiya, virusologiya 2003; (4):24-27 (in Russian)

### VII. Methods Based on Sequencing;

Kapur, V., L.-L. Li, S. Iordanescu, M. R. Hamrick, A. Wanger, B. N. Kre-iswirth, and J. M. Musser. 1994. Characterization by automated DNA sequencing of mutations in the gene (rpoB) encoding the RNA polymerase b subunit in rifampin-resistant Mycobacterium tuberculosis strains from New York City and Texas. J. Clin. Microbiol. 32:1095-1098.
Cavusoglu C, Karaca-Derici Y, Bilgic A. In-vitro activity of rifabutin against rifampicin-resistant Mycobacterium tuberculosis isolates with known rpoB mutations. Clin Microbiol Infect. 2004; 10(7):662-5.
   VIII. Dideoxy Fingerprinting (ddF-Method);
Rys P. N. and T. A. Felmlee. Detection of Rifampicin Resistance Mutations in Mycobacterium tuberculosis by Dideoxy Fingerprinting. In: PCR Protocols for Emerging Infectious Diseases (D. H. Persing, Ed.) (1996) ASM Press, Washington, pp. 112-121.

### IX. PCR-Heteroduplex Analysis;

Williams D. L., C. W. Limbers, L. Spring, S. Jayachandra, and T. P. Gillis. PCR-Heteroduplex Detection of Rifampicin-Resistant Mycobacterium tuberculosis. In: PCR Protocols for Emerging Infectious Diseases (D. H. Persing, Ed.) (1996) ASM Press, Washington, pp. 122-129.

### X. RNA Mismatch Analysis;

Dracopoli, N. C. Ed. “Detection of Mutations by RNase Cleavage”. Current Protocols in Human Genetics. 1998. John Wiley & Sons, Inc.
Marth G T, Korf I, Yandell M D et al. 1999. A general approach to single-nucleotide polymorphism discovery. Nat Genet., December; 23 (4):452-456.

### XI. Structure-Specific Endonuclease Cleavage;

Brow M. A. D., M. C. Oldenburg, V. Lyamichev, L. M. Heisler, N. Lyamicheva, J. G. Hall, N. J. Eagan, D. M. Olive, L. M. Smith, L. Fors, and J. E. Dahlberg. 1996. Differentiation of Bacterial 16S rRNA Genes and Intergenic Regions and Mycobacterium tuberculosis katG Genes by Structure-Specific Endonuclease Cleavage. J. Clin. Microbiol., 34: 3129-3137.

### XII. Line Probe Assay (LiPA);

Rossau R, Traore H, De Beenhouwer H, Mijs W, Jannes G, De Rijk P, Portaels F. Evaluation of the INNO-LiPA Rif. TB assay, a reverse hybridization assay for the simultaneous detection of Mycobacterium tuberculosis complex and its resistance to rifampin. Antimicrob Agents Chemother 1997; 41: 2093-2098.
Drobniewski F A, Watterson S A, Wilson S M, Harris G S. A clinical, microbiological and economic analysis of a national UK service for the rapid molecular diagnosis of tuberculosis and rifampin resistance in Mycobacterium tuberculosis. J Med Microbiol 2000; 49: 271-278.

### XIII. Bacteriophage-Based Method (PhaB Assay);

Wilson, S. M., Z. Al-Suwaidi, R. McNerney, J. Porter, and F. Drobniewski. 1997. Evaluation of a new rapid bacteriophage-based method for the drug susceptibility testing of Mycobacterium tuberculosis. Nat. Med. 3: 465-468.

### XIV. Cleavase Fragment Length Polymorphism (CFLP);

Sreevatsan S, Bookout J B, Ringpis F M, Mogazeh S L, Kreiswirth B N, Pottathil R R, Barathur R R. 1998. Comparative Evaluation of Cleavase Fragment Length Polymorphism With PCR-SSCP and PCR-RFLP to Detect Antimicrobial Agent Resistance in Mycobacterium tuberculosis. Mol Diagn. 1998 June; 3(2): 81-91.

### XV. Ligation-Based Assays;

Mikhailovich V., Lapa S., Gryadunov D., Sobolev A., Strizhkov B., Chernyh N., Skotnikova O., Irtuganova O., Moroz A., Litvinov V., Vladimirskii M., Perelman M., Chernousova L., Erokhin V., Zasedatelev A., and Mirzabekov A. 2001. Identification of rifampin-resistant Mycobacterium tuberculosis strains by hybridization, PCR, and ligase detection reaction on oligonucleotide microchips. J. Clin. Microbiol. 39: 2531-2540.

### XVI. Real-Time PCR.

Marin M, Garcia de Viedma D, Ruiz-Serrano M J, Bouza E. Rapid direct detection of multiple rifampin and isoniazid resistance mutations in Mycobacterium tuberculosis in respiratory samples by real-time PCR. Antimicrob Agents Chemother. 2004 November; 48(11):4293-300.
   XVII. Differential Sequencing with Mass Spectrometry;
Griffin, T. J. and L. M. Smith. 2000. Single nucleotide polymorphism analysis by MALDI-TOF mass spectrometry. Trends Biotechnol. 18:77-84.

### XVIII. Fluorescence Resonance Energy Transfer;

Viedma G. D., del Sol Diaz Infantes M, Lasala F, Chaves F, Alcala L, Bouza E. 2002. New Real-Time PCR Able To Detect in a Single Tube Multiple Rifampin Resistance Mutations and High-Level Isoniazid Resistance Mutations in Mycobacterium tuberculosis. J Clin Microbiol. 40(3):988-95.

### XIX. PCR on Oligonucleotide Microchips.

Mikhailovich V., Lapa S., Gryadunov D., Sobolev A., Strizhkov B., Chernyh N., Skotnikova O., Irtuganova O., Moroz A., Litvinov V., Vladimirskii M., Perelman M., Chernousova L., Erokhin V., Zasedatelev A., and Mirzabekov A. 2001. Identification of rifampin-resistant Mycobacterium tuberculosis strains by hybridization, PCR, and ligase detection reaction on oligonucleotide microchips. J. Clin. Microbiol. 39: 2531-2540.
Tillib S V, Strizhkov B N, Mirzabekov A D. Integration of multiple PCR amplifications and DNA mutation analyses by using oligonucleotide microchip. Anal Biochem. 2001 May 1; 292(1):155-60.

The following methods are used for detection of Mycobacterium tuberculosis complex:

### XX. Polymerase Chain Reaction for IS 6110 or 65 kD Heat Shock Protein;

Gunisha P, Madhavan H N, Jayanthi U, Therese K L. 2001. Polymerase chain reaction using IS6110 primer to detect Mycobacterium tuberculosis in clinical samples. Indian J Pathol Microbiol. 44(2):97-102.

### XXI. Ribosomal Intergenic Spacer Detection by Polymerase Chain Reaction;

Glennon M, Smith T, Cormican M, Noone D, Barry T, Maher M, Dawson M, Gilmartin J J, Gannon F. 1994. The ribosomal intergenic spacer region: a target for the PCR based diagnosis of tuberculosis. Tuber Lung Dis 75(5):353-60
Kraus G, Cleary T, Miller N, Seivright R, Young A K, Spruill G, Hnatyszyn H J. 2001. Rapid and specific detection of the Mycobacterium tuberculosis complex using fluorogenic probes and real-time PCR. Mol Cell Probes 15(6):375-83.
   XXII. Amplicor M. tuberculosis Test (ROCHE) for Amplifying Part of the 16S rRNA Gene.
J. Schirm, L. A. B. Oostendorp, and J. G. Mulder. 1995. Comparison of Amplicor, In-House PCR, and Conventional Culture for Detection of Mycobacterium tuberculosis in Clinical Samples. J. Clin. Microbiol. 33: 3221-3224.

### XXIII. Ligase Chain Reaction.

Viinanen A H, Soini H, Marjamaki M, Liippo K, Viljanen M K. 2000. Ligase chain reaction assay is clinically useful in the discrimination of smear-positive pulmonary tuberculosis from atypical mycobacterioses. Ann Med 32(4):279-283.
Lin I J, Che M J, Yeh A, Hwang J J, Wei C Y, Tsao W L, Lee C P. 1999. Comparison of the sensitivity and specificity of an automatic ligase chain reaction assay system with a one-step polymerase chain reaction assay in the diagnosis of Mycobacterium tuberculosis complex. Changgeng Yi Xue Za Zhi 22(2):204-211.

### XXIV. Real-Time PCR.

Shrestha N K, Tuohy M J, Hall G S, Reischl U, Gordon S M, Procop G W. Detection and differentiation of Mycobacterium tuberculosis and nontuberculous mycobacterial isolates by real-time PCR. J Clin Microbiol. 2003 November; 41(11):5121-5126.

The following disadvantages were noted for above mentioned methods for detection of mutations:
Methods of single nucleotide polymorphism and allele specific PCR in the test-tube (I, II) require the running of independent reactions for each mutation tested (i.e., more that 30 for rpoB gene) and respectively the big amount of sample for the study;
Methods of polymorphism analysis (III, IV), PCR-heteroduplex analysis (IX), and RNA mismatch analysis (X) are labour-intensive, time-consuming, provide indirect conclusion about type of mutation, and require typical standard (for each mutation); in addition, RNA mismatch analysis requires special conditions in terms of RNA-ase contamination, it is also labour-intensive and inapplicable for detection of G-U duplex (Nash, K. A., A. Gaytan, and C. B. Inderlied. 1997. Detection of rifampin resistance in Mycobacterium tuberculosis by means of a rapid, simple, and specific RNA/RNA mismatch assay. J. Infect. Dis. 176: 533-536; Telenti, A., P. Imboden, F. Marchesi, D. Lowrie, S. Cole, M. J. Colston, L. Matter, K. Schopfer, and T. Bodmer. 1993. Detection of rifampin resistance mutations in Mycobacterium tuberculosis. Lancet 341: 647-650);
Hybridization on high-density oligonucleotide microarrays (V) requires complicated computer-based data processing and expensive active storages (the microarray itself); now high-density microarrays are developed only for analysis of mutation in rpoB gene resulting in the resistance to rifampicin;
The known methods of hybridization on specialized microarrays (VI) are inapplicable for analysis of clinical samples because as test-samples they use DNA isolated from strain cultures that requires additional cultivation and purification of cells obtained from clinical material (Gryadunov D. A., Mikhailovich V. M., Lapa S. A., Roudinskii N. I., Barskii V. E., Chudinov A. V., Zasedatelev A. S., Mirzabekov A. D. Identification of Mycobacterium tuberculosis strains with simultaneous evaluation of their drug-resistance by hybridization method on oligonucleotide microarrays. Molekulyarnaya genetika, mikrobiologiya, virusologiya 2003; (4):24-27 (in Russian); Yue Jue, Wei Shi, Jingping Xie, Yao Li, Erliang Zeng and Honghai Wang. Detection of rifampin-resistant Mycobacterium tuberculosis strains by using a specialized oligonucleotide microarray. Diagn Microbiol Infect Dis 2004; 48: 47-54);
Direct nucleotide sequencing (VII) requires isolation of pure culture, running of amplification reaction and sequencing, and additional purification of reaction product for analysis using automatic sequencing machine;
Methods of dideoxy fingerprinting (VIII) and structure-specific endonuclease cleavage (XI) require preliminary standardization (selection of conditions) that increase labour-intensiveness, and require availability of typical standards. In addition, of dideoxy fingerprinting requires the use of radioactive probe that restricts its wide application in clinical laboratories;
Commercially available INNO-LiPA set (XII) has high cost price and provide identification of restricted number of mutations in rpoB gene (Rossau R, Traore H, De Beenhouwer H, Mijs W, Jannes G, De Rijk P, Portaels F. Evaluation of the INNO-LiPA Rif. TB assay, a reverse hybridization assay for the simultaneous detection of Mycobacterium tuberculosis complex and its resistance to rifampin. Antimicrob Agents Chemother 1997; 41: 2093-2098);
Bacteriophage-based method (XIII) is very time-consuming because it is connected with bacteriophage replication followed by lysis registration on culture of M. smegmatis, and is labour-intensive;
Methods of cleavase treatment (XIV) and mass-spectrometry (XVII) require homogeneous highly purified DNA sample; the stage of sample preparation and the high cost of equipment are also the limiting factors for wide application of mass-spectrometric analysis (XVII);
Methods based on ligation (XV) are used restrictedly because of difficulties with preparation of probe set for large number of detecting targets and because of use of expensive enzyme-thermo stable DNA-ligase;
Real-time PCR (XVI) reveals only the presence of the most spread mutations in tested genome fragment but does not allow precisely identify of nucleotide substitution and is relatively expensive for routine analysis;
Fluorescence resonance energy transfer method (XVIII) has two serious disadvantages: 1) it allows to detect only restricted number of mutations, and 2) it does not allow to differentiate the functionally significant mutations from mutations which are not phenotypically developed;
Methods of PCR and ligation on oligonucleotide microchips (XIX) require further improvement and are complicated for the application in practical laboratories.

All above mentioned methods for detection of Mycobacterium tuberculosis complex based on amplification of IS-element (XX), heat-shock protein (XX), ribosomal spacer gene detection (XXI), as well as methods of automatized PCR (XXII), ligase chain reaction (XXIII) and real-time PCR (XXIV) allow detect the presence of tuberculosis pathogen in tested sample but not the sensitivity of pathogens to antimycobacterial drugs.

### SUMMARY OF THE INVENTION

### Advantages of the Method for Detection of Strains of Mycobacterium tuberculosis Complex Resistant to Rifampicin and Isoniazid on Miniature Biochips

Method for detection of strains of Mycobacterium tuberculosis complex resistant to rifampicin and isoniazid in clinical samples on miniature biochips is favorably differed from methods known from state of the art by the ability to identify pathogen directly in clinical material with simultaneous evaluation of resistance to two antimycobacterial preparations of the first rank; by the ability to identify the type of mutation responsible for drug-resistance, as well as by low cost and short time necessary to obtain the result. The method does not require expensive equipment and highly skilled personnel. Data obtained with the use of the hybridization method can be used for determination therapeutic dosage of medical product and for epidemiological genetic typing.

In its first aspect the present invention provides the rapid method for simultaneous detection of Mycobacterium tuberculosis complex in clinical material and evaluation of the resistance of strains to rifampicin and isoniazid based on the identification of mutations in mycobacterial DNA resulting in the resistance of microorganisms to rifampicin and isoniazid with the use of miniature biochip. The method is based on two-stage multiplex PCR to obtain single-stranded fluorescent DNA fragments followed by hybridization of these fragments on microarray containing the set of differentiating oligonucleotides. The method includes the following stages:

(A)—multiplex amplification of fragments of rpoB, katG, inhA, ahpC genes, IS6110 mobile element with the use of set of pairs of specific primers for the first PCR stage;
(B)—multiplex amplification of fragments of rpoB, katG, inhA, ahpC genes, IS6110 mobile element with the use of PCR product obtained on the stage (A) as template, and set of pairs of specific primers for the second PCR stage, where one of primers in each pair is fluorescently labeled, to obtain predominantly single-stranded fluorescently labeled fragments;
(C)—preparation of biochip for simultaneous detection of Mycobacterium tuberculosis complex and identification of mutations resulting in resistance to rifampicin and isoniazid, which consists of matrix with gel pads where in each of pads the unique oligonucleotide probe is immobilized where the sequence of oligonucleotide is chosen from the group including the sequences: a) corresponding to sequence of fragment of wild type rpoB gene; b) corresponding to sequence of fragment of mutant variant rpoB gene resulting in resistance of microorganisms to rifampicin; c) complementary to sequences described in a) and b); d) corresponding to sequence of fragment of wild type katG gene; e) corresponding to sequence of fragment of mutant variant katG gene resulting in resistance of microorganisms to isoniazid; f) complementary to sequences described in d) and e); g) corresponding to sequence of fragment of wild type inhA gene; h) corresponding to sequence of fragment of mutant variant inhA gene resulting in resistance of microorganisms to isoniazid; i) complementary to sequences described in g) and h); j) corresponding to sequence of fragment of wild type ahpC gene; k) corresponding to sequence of fragment of mutant variant ahpC gene resulting in resistance of microorganisms to isoniazid; l) complementary to sequences described in j) and k); m) corresponding to sequence of mobile element IS6110; o) complementary to sequence described in m).
(D)—hybridization of amplified labeled products obtained on stage (B) on biochip under conditions providing the one nucleotide resolution between perfect and mismatched duplexes formed during hybridization;
(E)—registration and interpretation of the hybridization data.

In one of its embodiments the method is characterized by the use on the first stage of multiplex PCR (A) of the set of pairs of specific primers the sequences of which are presented by SEQ ID NO: 70, 71, 74, 75, 77, 78, 80, 81, 83, 84.

In another one of its embodiments the method is characterized by the use on the second stage of multiplex PCR (B) of the set of pairs of specific primers the sequences of which are presented by SEQ ID NO: 72, 73, 74, 76, 77, 79, 80, 82, 83, 85.

In the further embodiment the method is characterized by the use on the second stage of multiplex PCR (B) of the fluorescently labeled primer in molar excess in relation to second primer from this pair to obtain predominantly single-stranded fluorescently labeled fragments for all pairs of primers.

In the further embodiment the method is characterized by the conduction of amplification fragments of genes and IS6110 mobile element with the direct use of material from clinical sample (sputum, exudation, wash-out, bronchioalveolar lavage) or preliminary grown culture of microorganisms.

In another one of embodiments the method is characterized by the use of biochip containing the set of immobilized oligonucleotides the sequences of which are presented by SEQ ID NO: 1-69.

In the further embodiment the method is characterized by the use of hybridization buffer which allows conducting of hybridization in expanded temperature interval to provide the one nucleotide resolution between perfect and mismatched duplexes formed during hybridization.

In another one of embodiments the method is characterized by the registration of the data on stage (E) with the use of portable fluorescence analyzer and software that allows to conduct automatic processing of signal intensities with following interpretation of the data.

In another one of embodiments the method is characterized by the interpretation of the data obtained on stage (E) by the comparison of fluorescence signal intensities in the pads in which perfect and mismatched hybridization duplexes were formed.

Finally in another one of embodiments the method is characterized by the use of results of interpretation for confirmation of clinical diagnosis of tuberculosis and for epidemiological genetic typing using presence of one or another mutation as a marker.

In the following aspect the present invention provides the set of specific pairs of primers for realization of the first aspect of present invention, namely for the method for simultaneous detection of Mycobacterium tuberculosis complex and identification of mutations in mycobacterial DNA, resulting in the resistance of strains to rifampicin and isoniazid, in clinical samples where the sequences of primers are presented by SEQ ID NO: 70-85.

In another aspect the present invention provides the biochip which is used in the method according to the first aspect of the present invention, namely in the method for simultaneous detection of Mycobacterium tuberculosis complex and identification of mutations in mycobacterial DNA, resulting in the resistance of strains to rifampicin and isoniazid, in clinical samples, where the biochip consists of matrix with gel pads where in each of pads the unique oligonucleotide probe is immobilized, and where the sequences of probes are presented by SEQ ID NO: 1-69.

Finally, another one aspect of the present invention provides the set of oligonucleotide probes with sequences presented by SEQ ID NO: 1-69, which is used for preparation of biochip for the method of simultaneous detection of Mycobacterium tuberculosis complex and identification of mutations in mycobacterial DNA, resulting in the resistance of strains to rifampicin and isoniazid, in clinical samples according to the first aspect of the present invention.

Other aspects of the present invention will be seen from attached Figures, Detailed description of the invention, and Claims.

### BRIEF DESCRIPTION OF DRAWINGS

For the more clear understanding of the essence of the present invention as well as for demonstration of its characteristics features and advantages, the detailed description of the invention will be provided below with the references to the drawings in which:

FIG. 1 shows the scheme of arrangement of discriminating oligonucleotides on the biochip.

FIG. 2 shows hybridization pattern of DNA sample from wild type M. tuberculosis.

FIG. 3 shows hybridization pattern of DNA sample from M. tuberculosis containing the mutations resulting in amino acid substitutions Ser531>Leu in rpoB gene and Ser315>Thr in katG gene.

FIG. 4 shows hybridization pattern of DNA sample from M. tuberculosis containing the mutations resulting in amino acid substitution Asp516>Val in rpoB gene and nucleotide substitution C>T15 in promoter region of inhA gene.

FIG. 5 shows hybridization pattern of DNA sample of M. bovis BCG, which does not contain substitutions in rpoB and katG genes and in regulatory regions of inhA and ahpC genes.

FIG. 6 shows hybridization pattern of DNA sample of M. avium S58.

### DETAILED DESCRIPTION OF THE INVENTION

The aim of the present invention is to provide the rapid method of detection of Mycobacterium tuberculosis complex in clinical sample with simultaneous determination of resistance of mycobacterial to rifampicin and isoniazid by evaluation of minor polymorphism (pint mutations, deletions, insertions) in mycobacterial DNA resulting in appearance of drug-resistance.

The claimed method propose the use of: multiplex polymerase chain reaction for simultaneous amplification of sequences of mobile element IS 6110, rpoB, katG, inhA, ahpC genes; obtaining of fluorescently labeled single-stranded fragments of above mentioned genes with the use amplification products as template where the clinical material, lysate of cell culture, other physiological liquids could be used as a starting sample. The claimed method also provides the usage of original oligonucleotide biochip with immobilized specific probes, the procedures of hybridization, registration, and interpretation of the data.

Principal Scheme for Detection Rifampicin- and Isoniazid-Resistant Strains of Mycobacterium tuberculosis Complex on Biochip

Clinical sample (in the case of pulmonary form of disease—sputum) is exposed to decontamination and cell lysis to provide an access to genomic DNA. One of the suitable methods is dilution under alkaline conditions in the presence of N-acetyl-L-cysteine and boiling with detergent to provide an access to DNA and sample decontamination. For these purposes other approaches can be used as well which are known to specialists in the art such as cell destruction by ultrasound (Padilla E, Gonzalez V, Manterola J M, et al. Evaluation of two different cell lysis methods for releasing mycobacterial nucleic acids in the INNO-LiPA mycobacteria test. Diagn Microbiol Infect Dis. 2003 May; 46(1):19-23), lysis by guanidine thiocyanate-sarcosine (Kotlowski R, Martin A, Ablordey A et al. One-tube cell lysis and DNA extraction procedure for PCR-based detection of Mycobacterium ulcerans in aquatic insects, molluscs and fish. J Med Microbiol. 2004 September; 53(Pt 9):927-933), etc.

Specific amplification of fragments of functionally significant genes is carried out on the first stage using multiplex PCR. On the second stage predominantly single-stranded fluorescently labeled products are obtained by means of asymmetric multiplex PCR with the use of primers containing fluorescent dye on 5′-end.

Primers for the conducting of the first stage of amplification are chosen in order to they flank the gene region or regulatory region where the abundant mutations which lead to the drug-resistance of microorganism are located. Using special software, for example, Oligo v. 6.3 (Molecular Biology Insights Inc., CIIIA) or Fast PCR (http://www.biocenter.helsinki.fi/bi/Programs/fastper.htm) or other commercially available programs or programs available through the Internet, the melting temperatures of primers are calculated, and it is achieved by varying the length of primers that the dispersion of temperatures of annealing of primers will be not more than 3-4° C. During selection of primers it is necessary to avoid of such sequences which could form secondary structures like hair-pin with high melting temperatures. During selection of primers for multiplex PCR it is necessary to avoid of such sequences which could form duplexes between each other which are consisted of more than three-five nucleotides. Every selected primer should have unique specificity for analyzing region of the sequence of genome nucleic acids of Mycobacterium tuberculosis complex. Specificity of the primers is controlled by software which uses search in Databases of nucleotide sequences according BLAST algorithm (for example, www.ncbi.nlm.nih.gov/BLAST). Primers for simultaneous amplification of multiple genome regions are chosen if possible in such a way to provide the difference in the length of amplified fragments in 30-50 base pairs (b.p.) that allows a visual control of the presence/absence of specific band corresponding to the amplification product after electrophoresis in 2% agarose gel.

Primers for conducting of the second stage of amplification are chosen taking into account the above mentioned requests with the difference that the at least one of primers is chosen inside the PCR-fragment obtained on the first stage that increases the reaction specificity. Primers are chosen in such a way that the size of amplified fragments on the second stage will be 70-400 b.p. The greater length of PCR-products obtained on the second stage complicates efficient diffusion of the analyzed fragments in gel elements of biochip during hybridization that finally could lead to the decrease of the amount of formed hybridization duplexes and as a result to the decrease of fluorescence signal in the pads. During selection of primers it is necessary to take into account that predominantly single-stranded fluorescently labeled products are obtained on reaction yield which are complementary to oligonucleotides immobilized on biochip. Therefore in each pair the primer containing fluorescent label and which is added in an excess (“forward primer”) is chosen from the chain, the sequence of which is complementary to the sequences of oligonucleotides immobilized on biochip pads. I.e., in the case when oligonucleotides for immobilization are chosen from the sense chain, the predominant amplification of antisense chain is necessary for the formation of hybridization duplexes in biochip pads, thereby “forward primer” is chosen from the chain complementary to gene sequence (antisense chain) and vice versa.

Any fluorescent dye could be used as fluorescent label which can be chemically attached to 5′-end of oligonucleotide primer and which will not impede significantly the conducting of polymerase chain reaction. The spectrum of such dyes is well known to specialist in the art and includes, for example, the following dyes of fluorescene (TAMRA®, ROX®, JOE®), rodamine (Texas Red®), and polymethine (Cy3®, Cy5®, Cy5.5®, Cy7®) families (Ranasinghe R. and Brown T. Fluorescence based strategies for genetic analysis. Chem. Commun., 2005, 5487-5502). Fluorescent dyes are commercially available, in particular, from Molecular Probes, USA. The most preferable dyes have the excitation spectrum which is located in long-wave (red) region of spectrum that allows use of inexpensive light sources like semiconductor lasers for dye excitation.

Fluorescent dye can be attached to 5′-end of the primer either directly or via intermediate spacer, for example, 5′-Amino-Modifier from Glen Research. For fluorescent labeling of the primer the fluorescent dye is used in form of reactive derivative, for example, succinimide ether. Covalent modification of fluorescent dye can be carried out in manual mode after synthesis of primer and its separation from the solid matrix (CPG) on which synthesis was carried out. High performance liquid chromatography (HPLC) is used for purification of fluorescently labeled primer from unreacted dye and other side products of reaction.

For selection of discriminating oligonucleotides for immobilization on biochip the length of discriminating oligonucleotides is chosen in such a way which provides their specificity for analyzed sequence taking into account the size and complexity of analyzed sequence, in particular, the presence of repeats and extensive homopolymer sequences. For each position where mutations, polymorphism or allele variants are known the set of specific discriminating oligonucleotides is chosen, which is able to reveal known replacements, deletions, and insertions. The melting temperatures of oligonucleotides are calculated using software, for example, Oligo v. 6.3 (Molecular Biology Insights Inc., USA), and it is achieved by varying the length of primers that the dispersion of melting temperatures of oligonucleotides will be not more than 2-3° C. It is necessary to avoid of such oligonucleotides which could form secondary structures like hair-pin with high melting temperatures. The position of determinate variable nucleotides and other nucleotide reorganizations is chosen predominantly in location not far than 1-4 nucleotides from the middle of corresponding discriminating oligonucleotide.

Discriminating oligonucleotides are immobilized on gel elements which are deposited on matrix as the drops (pads) with diameter from 80 to 300 μm, spaced at 150-500 μm without the use of special devices, for example, quartz masks. The glass substrate (object-plate or cover glass) as well as more available materials such as plastic can be used as a matrix. For immobilization of oligonucleotides on biochips their copolymerization with main gel components is used. As a result of this one-stage reaction the immobilized molecules are irreversibly covalently attached to one or another monomers of the growing polymer chain and are uniformly distributed in whole gel volume with high yield (about 50% for oligonucleotides) (Rubina A Y, Pan'kov S V, Dementieva E I et al. Hydrogel drop microchips with immobilized DNA: properties and methods for large-scale production. Anal Biochem 2004; 325: 92-106). Concentration of immobilized oligonucleotide probes can be evaluated by staining of gel elements on biochip with a dye with low specificity to DNA nucleotide sequence (Mikheikin A. L., Chudinov A. V., Yaroshchuk A. I., Rubina A. Yu., Pan'kov S. V., Krylov A. C., Zasedatelev A. S., Mirzabekov A. D. The dye with low specificity to DNA nucleotide sequence: application for the evaluation of amount of oligonucleotides immobilized in array cells on biochips. Molekulyarnaya biologiya, 2003, 37(6): 1061-1070 (in Russian)).

Products of PCR obtained on the second stage of amplification are hybridized on differentiating biochip with immobilized oligonucleotides complimentary to the sequences of the tested genes y for detection of mutations. Hybridization is carried out in solution containing buffer component to keep pH, salt for making the ionic strength, and chaotropic agent (destabilizing hydrogen bonds) in hermetic hybridization camber at temperature which depends on the melting temperature of discriminating oligonucleotides immobilized on microarray. For example, guanidine thiocyanate, carbamide, or formamide can be used as agents destabilizing hydrogen bonds. Selection of optimal temperature for hybridization is carried out taking into account convenience of practical application of the system. Discriminating oligonucleotides claimed in the present invention have melting points in interval from 42 to 44° C. that allows to carry out the hybridization at 37° C. with the use of chaotropic agent. Temperature of 37° C. is convenient because the majority of clinical laboratories are equipped by thermostats maintaining this temperature. In the case when the developed method can be oriented for application of the system in a field condition the interval of temperatures used for hybridization can be expanded to 20-37° C. with corresponding change of the ionic strength of hybridization buffer from 0.3 to 1.0 M (Lapa S, Mikheev M, Shchelkunov S et al., Species-level identification of orthopoxviruses with an oligonucleotide microchip. J Clin Microbiol. 2002 March; 40(3):753-7).

The tested DNA fragments form perfect hybridization duplexes only with corresponding (totally complimentary) oligonucleotides. With all other oligonucleotides the tested DNA fragments form mismatched duplexes. Discrimination of perfect and mismatched duplexes is carried out by comparison of fluorescence intensity of array pads in which the duplexes were formed. Signal intensity in the pad where perfect hybridization duplex was formed (I_{perfect}) is higher than in the pad where mismatched duplex was formed (I_{mismatched}). The carrying out of hybridization under optimal conditions (temperature, appropriate concentration of chaotropic agent, and ionic strength of hybridization buffer) allows achieve the ratio I_{perfect}/I_{mismatched}≧2 between two pads containing probes which belong to one group and are different by one nucleotide.

Using the scheme of arrangement of oligonucleotides on biochip the mutations in tested DNA sequence are evaluated and conclusion about resistance or sensitivity of tested sample to rifampicin and/or isoniazid is made.

The intensities of fluorescent signals in the gel pads belonging to one group are compared between each other. Maximal fluorescent signal testifies for formation of perfect hybridization duplex in the pad in which this signal was registered. If maximal signal was registered in the pad corresponding to DNA without mutations (i.e., belonging to microorganism sensitive to the medical drug) it is suggested that tested sample does not have mutations in this amino acid position (group of gel pads). If maximal signal was registered in the gel pad corresponding to DNA with mutation (mutations) (i.e., belonging to microorganism resistant to the medical drug) it is suggested that the tested sample has amino acid replacement resulting in drug-resistance in this amino acid position (group of gel pads). The tested sample is considered as belonging to sensitive strain of mycobacteria if the sample was characterized as sensitive in each amino acid position (group of pads). The tested sample is considered as belonging to resistant strain of mycobacteria if the sample was characterized as having mutation resulting in drug-resistance in at least one amino acid position (group of gel pads). For such samples the type of drug to which resistance was found is additionally elucidated by determination of the group in which the sample belongs to drug-resistant type.

The group consisting of two pads forms separately the system for detection of Mycobacterium tuberculosis complex DNA. Interpretation of the data in this system is based on registration of more intensive fluorescent signal in detecting pads in comparison with reference pads. The gel pads without immobilized oligonucleotides are used as reference pads. If more intensive signals are registered in detecting pads the conclusion is made that the tested sample contains DNA belonging to Mycobacterium tuberculosis complex.

The following examples are given and served to further illustrate and describe the scope and details of the present invention, which shall not represent any limitation to this invention.

### EXAMPLES

### Example 1

### Biochip for Detection of Mycobacterium tuberculosis Complex and for Evaluation of the Sensitivity of Strains to Rifampicin and Isoniazid

1. Oligonucleotides for immobilization on biochip and primers for amplification were synthesized on automatic synthesizer 394 DNA/RNA synthesizer (Applied Biosystems, USA) and contained spacer with free amino group 3′-Amino-Modifier C7 CPG 500 (Glen Research, USA) for further immobilization in gel or 5′-Amino-Modifier C6 (Glen Research, USA) for attachment of fluorescent dye, respectively. Attachment of indodicarbocyanine fluorescent dye (“Biochip-IMB”, Russia) was carried out according to the recommendations of manufacturer. Biochips were manufactured according to the procedure described earlier (Rubina A Y, Pan'kov S V, Dementieva E I et al. Hydrogel drop microchips with immobilized DNA: properties and methods for large-scale production. Anal Biochem 2004; 325: 92-106). Biochips contained semispherical pads with diameter of 100 μm, spaced at 300 μm from each other. Uniformity of pad deposition and their diameter were evaluated by “Test-chip” software (“Biochip-IMB”, Russia). Control of microarray quality was carried out by the measuring of concentrations of immobilized oligonucleotides. Biochips were stained by fluorescent dye ImD-310 and concentration of immobilized probes was measured as described earlier (Mikheikin A. L., Chudinov A. V., Yaroshchuk A. I., Rubina A. Yu., Pan'kov S. V., Krylov A. C., Zasedatelev A. S., Mirzabekov A. D. The dye with low specificity to DNA nucleotide sequence: application for the evaluation of amount of oligonucleotides immobilized in array pads on biochips. Molekulyarnaya biologiya, 2003, 37(6): 1061-1070 (in Russian)).

Biochip Structure

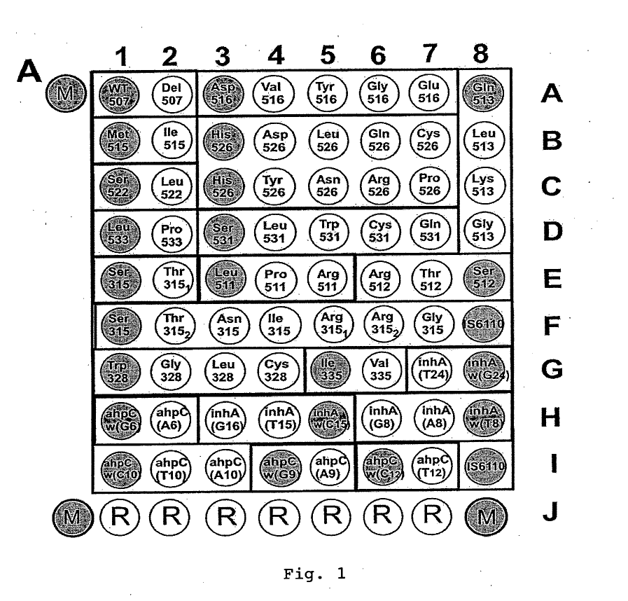

Biochip contains 69 immobilizes oligonucleotides listed in Table 1, three marker points for correct positioning (image capture) provided by software, and 7 reserve pads of blank gel. Arrangement of oligonucleotides immobilized on microarray is shown on FIG. 1.

Gray color on FIG. 1 indicates the gel pads containing oligonucleotides (Table 1, SEQ ID NO 1-69), which are able to form perfect hybridization duplex with DNA without mutations (i.e., with wild type (WT) DNA) in positions corresponding to the following amino acid residues or nucleotides in promoter region of the genes:

rpoB: (Gly/Thr) WT507 (A1); Met515 (B1); Ser522 (C1); Leu533 (D1); Asp516 (A3); His (B3, C3); Ser531 (D3); Leu511 (E3); Gln513 (A8); Ser512 (E8);

katG: Ser315 (E1,F1); Trp328 (G1); Ile335 (G5);

inhA: inhAw_G24 (G8); inhAw_AT8 (H8); inhAw_C15 (H5);

ahpC: ahpCw_G6 (H1); ahpCw_C10 (I1); ahpCw_G9 (I4); ahpCw_C12 (I6).

White color on FIG. 1 indicates the gel pads containing oligonucleotides which form mismatched hybridization duplexes with wild type DNA, in positions corresponding to the following amino acid residues or nucleotides in promoter region of the genes:

rpoB: WT507 (A2); Ile515 (B2); Leu522 (C2); Pro533 (D2); VA1516 (A4); Tyr516 (A5); Gly516 (A6); Glu516 (A7); Asp526 (B4); Leu526 (B5); Gln526 (B6); Cys526 (B7); Tyr526 (C4); Asn526 (C5); Arg526 (C6); Pro526 (C7); Leu531 (D4); Trp531 (D5); Cys531 (D6); Gln531 (D7); Pro511 (E4); Arg511 (E5); Thr512 (E7); Arg512 (E6); Leu513 (B8); Lys513 (C8); Gly513 (D8);

TABLE 1
List of sequences of oligonucleotides immobilized on biochip.

| SEQ | | Position | | | | Position of |
|---|---|---|---|---|---|---|
| ID | Oligo- | of amino | Substitution of | Substitution of | | oligonucleotide |
| NO: | nucleotide* | acid/nucleotide | amino acid | nucleotide | Sequence 5′→3′ | in gene sequence |
| | | | | | Sequence of rpoB | |
| | | | | | gene (Genbank Acc. | rpoB/Acc. |
| | | | | | No L27989) | No L27989 |
| 1 | A1 | 507 | WT | — | TGG CTG GTG CCG AAG A | 2370-2353 |
| 2 | A2 | 507 | DEL | GGCACC | CTC AGC TGG CTG AAG AA | 2376-2352 |
| 3 | E3 | 511 | Leu (WT) | CTG | TTG GCT CAG CTG GCT G | 2380-2364 |
| 4 | E4 | 511 | Leu > Pro | CTG > CCG | TTG GCT CGG CTG GCT | 2380-2364 |
| 5 | E5 | 511 | Leu > Arg | CTG > CGG | TTG GCT CCG CTG GCT | 2380-2364 |
| 6 | E8 | 512 | Ser (WT) | AGC | AAT TGG CTC AGC TGG C | 2383-2365 |
| 7 | E7 | 512 | Ser > Thr | AGC > ACC | AAT TGG GTC AGC TGG | 2383-2365 |
| 8 | E6 | 512 | Ser > Arg | AGC > CGC | AAT TGG CGC AGC TG | 2383-2367 |
| 9 | A8 | 513 | Gln (WT) | CAA | TCC ATG AAT TGG CTC | 2386-2368 |
| | | | | | AGC T | |
| 10 | B8 | 513 | Gln > Leu | CAA > CTA | CAT GAA TAG GCT CAG CT | 2386-2368 |
| 11 | C8 | 513 | Gln > Lys | CAA > AAA | CAT GAA TTT GCT CAG CT | 2386-2368 |
| 12 | D8 | 513 | Gln > Pro | CAA > CCA | CAT GAA TGG GCT CAG CT | 2386-2368 |
| 13 | B1 | 515 | Met (WT) | ATG | TTC TGG TCC ATG AAT | 2396-2374 |
| | | | | | TGG | |
| 14 | B2 | 515 | Met > Ile | ATG > ATA | GTT CTG GTC TAT GAA | 2396-2374 |
| | | | | | TTG G | |
| 15 | A3 | 516 | Asp (WT) | GAC | TT GTT CTG GTC CAT GAA | 2397-2376 |
| | | | | | T | |
| 16 | A4 | 516 | Asp > Val | GAC > GTC | TT GTT CTG GAC CAT GAA | 2397-2376 |
| | | | | | T | |
| 17 | A5 | 516 | Asp > Tyr | GAC > TAC | TT GTT CTG GTA CAT GAA | 2397-2376 |
| | | | | | T | |
| 18 | A6 | 516 | Asp > Gly | GAC > GGC | TT GTT CTG GCC CAT GAA | 2397-2376 |
| | | | | | T | |
| 19 | A7 | 516 | Asp > Glu | GAC > GAG | TT GTT CTG CTC CAT GAA | 2397-2376 |
| | | | | | T | |
| 20 | C1 | 522 | Ser (WT) | TCG | T CAA CCC CGA CAG CG | 2414-2398 |
| 21 | C2 | 522 | Ser > Leu | TCG > TTG | T CAA CCC CAA CAG CG | 2414-2398 |
| 22 | B3,C3 | 526 | His (WT) | CAC | G GCG CTT GTG GGT CAA | 2424-2408 |
| | | | | | C | |
| 23 | B4 | 526 | His > Asp | CAC > GAC | G GCG CTT GTC GGT CAA | 2424-2408 |
| | | | | | C | |
| 24 | B5 | 526 | His > Leu | CAC > CTC | G GCG CTT GAG GGT CAA | 2424-2408 |
| | | | | | C | |
| 25 | B6 | 526 | His > Gln | CAC > CAA | G GCG CTT TTG GGT CAA | 2424-2408 |
| | | | | | C | |
| 26 | B7 | 526 | His > Cys | CAC > TGC | G GCG CTT GCA GGT CAA | 2424-2408 |
| | | | | | C | |
| 27 | C4 | 526 | His > Tyr | CAC > TAC | G GCG CTT GTA GGT CAA | 2424-2408 |
| | | | | | C | |
| 28 | C5 | 526 | His > Asn | CAC > AAC | G GCG CTT GTT GGT CAA | 2424-2408 |
| | | | | | C | |
| 29 | C6 | 526 | His > Arg | CAC > CGC | G GCG CTT GCG GGT CAA | 2424-2408 |
| | | | | | C | |
| 30 | C7 | 526 | His > Pro | CAC > CCC | G GCG CTT GGG GGT CAA | 2424-2408 |
| | | | | | C | |
| 31 | D3 | 531 | Ser (WT) | TCG | CAG CGC CGA CAG TCG | 2438-2424 |
| 32 | D4 | 531 | Ser > Leu | TCG > TTG | C CAG CGC CAA CAG TCG | 2439-2424 |
| 33 | D5 | 531 | Ser > Trp | TCG > TGG | CAG CGC CCA CAG TCG | 2438-2424 |
| 34 | D6 | 531 | Ser > Cys | TCG > TGT | CAG CGC ACA CAG TCG | 2438-2424 |
| 35 | D7 | 531 | Ser > Gln | TCG > CAG | CAG CGC CTG CAG TCG | 2438-2424 |
| 36 | D1 | 533 | Leu (WT) | CTG | TG CCC CAG CGC CGA CAG | 2443-2427 |
| 37 | D2 | 533 | Leu > Pro | CTG > CCG | TG CCC CGG CGC CGA CAG | 2443-2427 |
| | | | | | Sequence of katG | |
| | | | | | gene (Genbank Acc. | katG/Acc. |
| | | | | | No U06262) | No U06262 |
| 38 | E1, F1 | 315 | Ser (WT) | AGC | CG ATC ACC AGC GGC AT | 1004-1019 |
| 39 | E2 | 315 | Ser > Thr₁ | ACC | CG ATC ACC ACC GGC AT | 1004-1019 |
| 40 | F2 | 315 | Ser > Thr₂ | ACA | CG ATC ACC ACA GGC ATC | 1004-1020 |
| 41 | F3 | 315 | Ser > Asn | AAC | G ATC ACC AAC GGC ATC | 1005-1020 |
| 42 | F4 | 315 | Ser > Ile | ATC | G ATC ACC ATC GGC ATC | 1005-1020 |
| 43 | F5 | 315 | Ser > Arg₁ | CGC | G ATC ACC CGC GGC AT | 1005-1020 |
| 44 | F6 | 315 | Ser > Arg₂ | AGA | ATC ACC AGA GGC ATC G | 1006-1021 |
| 45 | F7 | 315 | Ser > Gly | GGC | G ATC ACC GGC GGC AT | 1005-1019 |
| 46 | G1 | 328 | Trp (WT) | TGG | CG AAA TGG GAC AAC A | 1046-1060 |
| 47 | G2 | 328 | Trp > Gly | GGG | CG AAA GGG GAC AAC A | 1046-1060 |
| 48 | G3 | 328 | Trp > Leu | TTG | CG AAA TTG GAC AAC AG | 1046-1060 |
| 49 | G4 | 328 | Trp > Cys | TGC | CG AAA TGC GAC AAC A | 1046-1060 |
| 50 | G5 | 335 | Ile (WT) | ATC | TC CTC GAG ATC CTG TAC | 1064-1081 |
| | | | | | G | |
| 51 | G6 | 335 | Ile > Val | GTC | TC CTC GAG GTC CTG TAC | 1064-1081 |
| | | | | | G | |
| | | | | | Sequence of inhA | |
| | | | | | gene (Genbank Acc. | inhA/Acc. |
| | | | | | No AY192027) | AY192027 |
| 52 | G8 | 24 | — | G(WT) | CCG GCC GCG GCG | 164-175 |
| 53 | G7 | 24 | — | G > T | CCG GCC TCG GCG | 164-175 |
| 54 | H8 | 8 | — | T(WT) | GAT AGG TTG TCG GGG | 180-194 |
| 55 | H7 | 8 | — | T > A | GAT AGG ATG TCG GGG | 180-194 |
| 56 | H6 | 8 | — | T > G | GAT AGG GTG TCG GGG | 180-194 |
| 57 | H5 | 15 | — | C(WT) | CGG CGA GAC GAT AGG | 171-185 |
| 58 | H4 | 15 | — | C > T | CGG CGA GAT GAT AGG | 171-181 |
| 59 | H3 | 16 | — | C > G | GG CGA GGC GAT AGG | 172-185 |
| | | | | | Sequence of ahpC | |
| | | | | | gene (Genbank Acc. | ahpC/Acc. |
| | | | | | No U16243) | No U16243) |
| 60 | H1 | 6 | — | G(WT) | GCA CGA TGG AAT GTC GC | 680-696 |
| 61 | H2 | 6 | — | G > A | GCA CGA TAG AAT GTC | 679-696 |
| | | | | | GCA | |
| 62 | I1 | 10 | — | C(WT) | TCA CGG CAC GAT GGA A | 686-701 |
| 63 | I2 | 10 | — | C > T | TCA CGG CAT GAT GGA A | 686-701 |
| 64 | I3 | 10 | — | C > A | TCA CGG CAA GAT GGA A | 686-701 |
| 65 | I4 | 9 | — | G(WT) | CAC GGC ACG ATG GAA T | 685-700 |
| 66 | I5 | 9 | — | G > A | CAC GGC ACA ATG GAA T | 685-700 |
| 67 | I6 | 12 | — | C(WT) | CT TCA CGG CAC GAT GG | 688-703 |
| 68 | I7 | 12 | — | C > T | ACT TCA CGG TAC GAT | 687-704 |
| | | | | | GGA | |
| | | | | | Sequence of IS6110 | |
| | | | | | element (Genbank Acc. | IS6110/Acc. |
| | | | | | No AF189827) | No AF189827 |
| 69 | F8, I8 | — | — | WT | CCG GAG CTG CGT GAG CG | 1359-1375 |

*Positions of oligonucleotides are shown in accordance with their location on FIG. 1.

katG: Thr315₁ (E2); Thr315₂ (F2); Asn315 (F3); Ile315 (F4); Arg315₁ (F5); Arg315₂ (F6); Gly315 (F7); Gly328 (G2); Leu328 (G3); Cys328 (G4); Val335 (G6);

inhA: inhA_T24 (G7); inhA_A8 (H7); inhA_G8 (H6); inhA_T15 (H4); inhA_G16 (H3);

ahpC: ahpC_A6 (H2); ahpC_T10 (I2); ahpC_A10 (I3); ahpC_A9 (I5); ahpC_T12 (I7).

Pads F8 and I8 contain oligonucleotide complementary to fragment of the sequence of insertion element IS6110 which as a rule is present in DNA of Mycobacterium tuberculosis complex.

### Example 2

### Treatment of Clinical Sample

1. Clinical sample (sputum, exudation, wash-out, bronchioalveolar lavage) was mixed in 1:1 (v/v) ratio with freshly prepared 0.5% solution of N-acetyl-L-cysteine (NALC) in 2% NaOH. Sample was rigorously stirred by Vortex and kept at room temperature for 20 min. Phosphate buffer saline pH 6.8 was added to the sample in ratio 1:5 (v/v) and mixture was centrifuged for 30 min at 3,000 rpm. When cerebrospinal fluid was used the preliminary centrifugation for 10 min at 10,000 rpm was carried out. For blood analysis lymphocyte fraction was preliminarily isolated according common method with Ficoll. Subsequent treatment of all samples was carried out identically.
2. Precipitated pads were suspended in 1.5 ml of TE buffer (10 mM Tris-HCl, 1 mM EDTA), pH 8.0, and centrifuged at 3,000 rpm for 30 min. The washing procedure was repeated one more time.
3. To the pellet obtained 30 μl of TE buffer, pH 8.0, containing 1% (v/v) Triton X-100 was added and sample was kept in dry oven at 95° C. for 30 min.
4. The sample was centrifuged at 10,000 rpm for 10 min and supernatant (3 μl) was used for PCR.

### Example 3

### Amplification of Fragments of IS6110 Mobile Element, rpoB, katG, inhA, ahpC Genes; Preparation of Single-Stranded Fluorescently Labeled Fragments by the Method of Multiplex PCR

On the first stage the multiplex amplification of fragments of rpoB (212 b.p.), katG (166 b.p.), inhA (133 b.p.), ahpC (126 b.p.) genes and of IS6110 mobile element (309 b.p) was carried out.

Into 25 μl of PCR-mixture the 3 μl of sample obtained in paragraph 4 of Example 2 were added.

Composition of PCR-mixture:
1×PCR-buffer: 10 mM KCl, 10 mM Tris-HCl (pH 8.3) (Sileks, Russia);
1.5 mM MgCl₂
200 μM of each of dATP, dCTP, dGTP, dUTP (Sileks)
The mixture of primers (sequences are listed in Table 2) in following concentration: p105f, p293r, katG_f, katG_r1, IS_f, IS_r1; InhA_f, InhA_r1, ahpc_f, ahpC_r1—100 nM.
5 U of thermostable Taq DNA-polymerase (Sileks)
0.5 U of uracyl-DNA-glycosylase (Sileks)

Amplification was carried out on programmable thermostat MiniCycler (MJ Research, USA) under following conditions: 95° C.-30 s, 67° C.-30 s, 72° C.-30 s; 36 cycles (in the first cycle the time of denaturation was increased to 5 min, in final cycle the time of fitting-out was increased to 5 min).

As a template for the second stage 1 μl of reaction mixture obtained after first stage of PCR was used.

The second stage of PCR was carried out with primers specific to fragments of rpoB (126 b.p.), katG (140 b.p.), inhA (93 b.p.), ahpC (96 b.p.) genes and IS6110 (110 b.p.)

Composition of PCR-mixture (50 μl):
1×PCR-buffer: 10 mM KCl, 10 mM Tris-HCl (pH 8.3) (Sileks, Russia);
1.5 mM MgCl₂
200 μM of each of dNTP (Sileks)
The mixture of primers (sequences are listed in Table 2) in following concentration: p1272f*/p1398r, 100 nM; katG_f/katG_r2*, 100 nM; inhA_f/inhA_r2*, 100 nM; ahpC_f/ahpC_r2*, 100 nM; IS_f/IS_r2*, 100 nM.
10 U of thermostable Taq DNA-polymerase (Sileks)

5′-End of primers indicated by * contained linker C₆ aminomodifier (Glen Research Corp., USA) to amino group of which indodicarbocyanine dye (Biochip-IMB, Moscow) was attached.

Amplification was carried out on programmable thermostat MiniCycler (MJ Research, USA) under following conditions: 95° C.-30 s, 65° C.-30 s, 72° C.-20 s; 46 cycles (in the first cycle the time of denaturation was increased to 5 min, in final cycle the time of fitting-out was increased to 5 min). The obtained product (12 μl) was used for hybridization on biochip.

### Example 4

### Hybridization of Amplified Labeled Products on Biochip

To 12 μl of reaction mixture obtained after second stage of PCR and containing predominantly single-stranded fluorescently labeled DNA fragments which correspond to five analyzed fragment, concentrated solution of hybridization buffer was added to achieve final

TABLE 2

List of sequences of primers used in two-stage PCR for amplification of fragments of
rpoB, katG, inhA, ahpC genes and fragment of element IS 6110.

| SEQ | | | Location of primer in gene | Gene/ |
|---|---|---|---|---|
| ID NO: | Name | Sequence 5′→3′ | sequence | GenBank Acc. No |
| 70 | p105f | CGTGGAGGCGATCACACCGCAGACGTTG | 2288-2315 | rpoB/L27989 |
| 71 | p293r | AGTGAGACGGGTGCACGTCGCGGACCT | 2476-2503 | rpoB/L27989 |
| 72 | P1272f* | CGCCGCGATCAAGGAGTTCT | 2336-2355 | rpoB/L27989 |
| 73 | p1398r | TCACGTGACAGACCGCCGGG | 2442-2461 | rpoB/L27989 |
| 74 | katG_f | TGGAAGAGCTCGTATGGCACCGGAACC | 967-993 | katG/U06262 |
| 75 | katG_r1 | CGGTGTATTGCCAAGCGCCAGCAG | 1109-1132 | katG/U06262 |
| 76 | katG_r2* | GCTCTCCGTCAGCTCCCACTCGTAGCC | 2990-3016 | katG/U06262 |
| 77 | inhA_f | CCGGAAATCGCAGCCACGTTACGC | 118-141 | inhA/AY192027 |
| 78 | inhA_r1 | GGTAACCAGGACTGAACGGGATACGAA | 230-256 | inhA/AY192027 |
| 79 | inhA_r2* | GGCCCCTTCAGTGGCTGTGGCAG | 198-220 | inhA/AY192027 |
| 80 | ahpC_f | ATGGTGTGATATATCACCTTTGCCTGACAGC | 706-736 | ahpC/U16243 |
| 81 | ahpC_r1 | GGAATTGATCGCCAATGGTTAGCAGTGG | 611-638 | ahpC/U16243 |
| 82 | ahpC_r2* | TGACTCTCCTCATCATCAAAGCGGACA | 641-667 | ahpC/U16243 |
| 83 | IS_f | AGGATGGGGTCATGTCAGGTGGTTCATCGA | 1318-1347 | IS6110/AF189827 |
| 84 | IS_r1 | GCGAAGAAAGCCGACGCGGTCTTTAAAATC | 1598-1627 | IS6110/AF189827 |
| 85 | ISr2* | CGCTGCCCACTCCGAATCGTGC | 1409-1430 | IS6110/AF189827 |

*5′-end of primers contained linker C₆ aminomodifier (Glen Research Corp., USA) to amino group of which indodicarbocyanine dye IMD-504 (Biochip-IMB, Moscow) was attached.

concentrations of guanidine thiocyanate—1M, HEPES—50 mM, pH 7.5, EDTA—5 mM. The mixture obtained (28 μl) was placed into hybridization chamber of microarray. The cambers were manufactured by “Biochip-IMB” Ltd. (Russia). Hybridization was carried out at 37° C. for 6-12 hours. After that the microarray was washed three times by distilled water at 37° C.

### Example 5

### Registration and Interpretation of Hybridization Data

Registration of fluorescent image of biochip was carried out on portable fluorescence analyzer (“Biochip-IMB” Ltd., Russia) using ImageWare® software developed by the same Company.

Interpretation of the data was carried out in the following way. Gel pads containing oligonucleotides are clustered into 22 groups (these groups are divided by solid lines on FIG. 1) in such a way that the comparison of intensities of fluorescent signals within each group allows to make conclusion about presence/absence of mutation (minor polymorphism) resulting in substitution of single amino acid residue or deletion, or nucleotide substitution in promoter region of inhA and ahpC genes.

### Example 6

### Detection of Mycobacterium tuberculosis Complex Sensitive to Rifampicin and Isoniazid (Wild Type DNA) in Clinical Sample Using Method of Hybridization on Biochip

The sample of sputum obtained from patient with confirmed (by microscopy) presence of acid-fast bacteria (AFB-positive) was divided into 2 parts one of which after decontamination (N-acetyl-L-cysteine and NaOH) and neutralization was inoculated on Lowenstein-Jensen medium. The sample was incubated at 37° C. for 6 weeks and growth of bacterial colonies was controlled weekly. After discovering of the colonies the presence of acid-fast bacteria in the sample was revealed by microscopy after Ziehl-Neelsen staining. Identification of Mycobacterium tuberculosis complex was carried out by biochemical tests (Kent P T, Kubica G P. Public health mycobacteriology. A guide for level III laboratory. Atlanta, Ga.: Centers for Disease Control and Prevention, 1985). Tests on drug-resistance were carried out by the proportion method on Lowenstein-Jensen media containing rifampicin and isoniazid at concentrations 40 mg/l and 0.2 mg/l, respectively. The isolate was considered as resistant if more that 1% of colonies grew on the drug-containing medium compared with drug-free medium.

The second part of sputum sample was used for analysis by the method of the present invention as described in Examples 2-5.

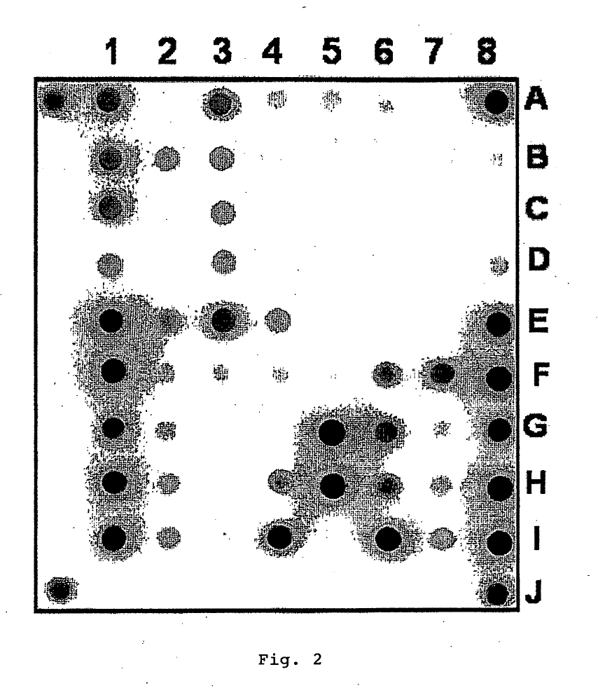

FIG. 2 shows the data of hybridization of DNA sample isolated from sensitive to rifampicin and isoniazid strain of Mycobacterium tuberculosis complex.

According to proposed algorithm of data interpretation the pads with oligonucleotides complimentary to wild type DNA have higher intensity of fluorescent signal in comparison with other pads inside each group. Therefore DNA of tested sample forms perfect hybridization duplexes with oligonucleotides complimentary to the sequence of wild type DNA (strain of tuberculosis mycobacteria sensitive to rifampicin and isoniazid).

Belonging of the strain to Mycobacterium tuberculosis complex is established by comparison of intensity of fluorescent signal in the pads with immobilized oligonucleotide complimentary to IS6110 (F8, I8) and in blank pads which play a role of negative control.

It means that Mycobacterium tuberculosis complex is present in the tested clinical sample and that the bacteria resistant to rifampicin and isoniazid are absent.

The presence of M. tuberculosis in sputum samples was confirmed by the cultivation on solid media and by biochemical tests. The growth of colonies on the media with drugs was absent. Therefore, the presence of wild type M. tuberculosis in clinical samples was revealed by cultural method that is in a whole agreement with the data obtained by the method of the present invention.

### Example 7

### Detection of Mycobacterium tuberculosis Complex Resistant to Rifampicin and Isoniazid (DNA Containing Mutations) in Clinical Sample by the Method of Hybridization on Biochip

The sample of sputum obtained from patient with confirmed (by microscopy) presence of acid-fast bacteria (AFB-positive) was divided into 2 parts one of which after decontamination (N-acetyl-L-cysteine and NaOH) and neutralization was inoculated on Lowenstein-Jensen medium. The sample was incubated at 37° C. for 6 weeks and growth of bacterial colonies was controlled weekly. After discovering of the colonies the presence of acid-fast bacteria in the sample was revealed by microscopy after Ziehl-Neelsen staining. Identification of Mycobacterium tuberculosis complex was carried out by biochemical tests (Kent P T, Kubica G P. Public health mycobacteriology. A guide for level III laboratory. Atlanta, Ga.: Centers for Disease Control and Prevention, 1985). Tests on drug-resistance were carried out by the proportion method on Lowenstein-Jensen media containing rifampicin and isoniazid at concentrations 40 mg/l and 0.2 mg/l, respectively. The isolate was considered as resistant if more that 1% of colonies grew on the drug-containing medium compared with drug-free medium.

The second part of sputum sample was used for analysis by the method of the present invention as described in Examples 2-5.

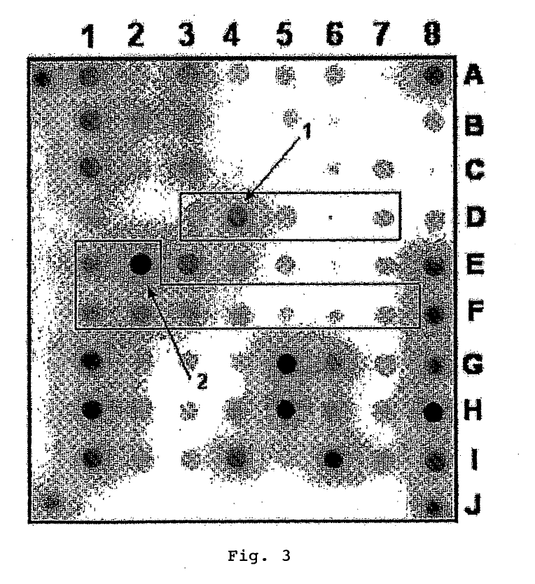

FIG. 3 shows the data of hybridization of DNA sample isolated from resistant to rifampicin and isoniazid strain of Mycobacterium tuberculosis complex.

In each group of biochip elements fluorescence intensity in the pad with oligonucleotide complimentary to wild type DNA is higher than the signal intensity in all other pads. Only groups of elements D3-D7 and E1, E2, F1-F7 are the exception (FIG. 3).

Element D4 demonstrates maximal fluorescence intensity within the group D3-D7. Therefore, DNA of the tested sample contains point nucleotide substitution A>T in the position 2431 of rpoB gene (Genbank Acc. No L27989) that leads to amino acid substitution Ser to Leu in position No. 531 and results in the resistance of the tested strain to rifampicin.

Element E2 demonstrates maximal fluorescence intensity within group E1,E2, F1-F7. Therefore, the tested DNA contains point nucleotide substitution G>C in the position 1013 of katG gene (Genbank Acc. No U06262) that leads to amino acid substitution Ser315 to Thr in position No. 315 and results in the resistance of the tested strain to isoniazid.

Belonging of this strain to Mycobacterium tuberculosis complex is established by comparison of intensity of fluorescent signal in the pads with immobilized oligonucleotide complimentary to IS6110 (F8, I8) and in blank pads which play a role of negative control.

Therefore, it is concluded that Mycobacterium tuberculosis complex resistant to rifampicin and isoniazid is present in the tested clinical sample. Resistance to rifampicin is the result of amino acid substitution Ser to Leu in the position No. 531 of rpoB gene. Resistance to isoniazid is the result of amino acid substitution Ser315 to Thr in katG gene.

The presence of M. tuberculosis in sputum sample was confirmed by the cultivation on solid media and by biochemical tests. Therefore, the presence of drug-resistant M. tuberculosis was confirmed by the stable growth of colonies on the media with rifampicin and isoniazid that is in a whole agreement with the data obtained by the method of the present invention.

### Example 8

### Detection of Mycobacterium tuberculosis Complex Resistant to Rifampicin and Isoniazid (DNA with Mutations) in Clinical Sample by the Method of Hybridization on Biochip

The sample of sputum obtained from patient with confirmed (by microscopy) presence of acid-fast bacteria (AFB-positive) was divided into 2 parts one of which after decontamination (N-acetyl-L-cysteine and NaOH) and neutralization was inoculated on Lowenstein-Jensen medium. The sample was incubated at 37° C. for 6 weeks and growth of bacterial colonies was controlled weekly. After discovering of the colonies the presence of acid-fast bacteria in the sample was revealed by microscopy after Ziehl-Neelsen staining. Identification of Mycobacterium tuberculosis complex was carried out by biochemical tests (Kent P T, Kubica G P. Public health mycobacteriology. A guide for level III laboratory. Atlanta, Ga.: Centers for Disease Control and Prevention, 1985). Tests on drug-resistance were carried out by the proportion method on Lowenstein-Jensen media containing rifampicin and isoniazid at concentrations 40 mg/l and 0.2 mg/l, respectively. The isolate was considered as resistant if more that 1% of colonies grew on the drug-containing medium compared with drug-free medium.

The second part of sputum sample was used for analysis by the method of the present invention as described in Examples 2-5.

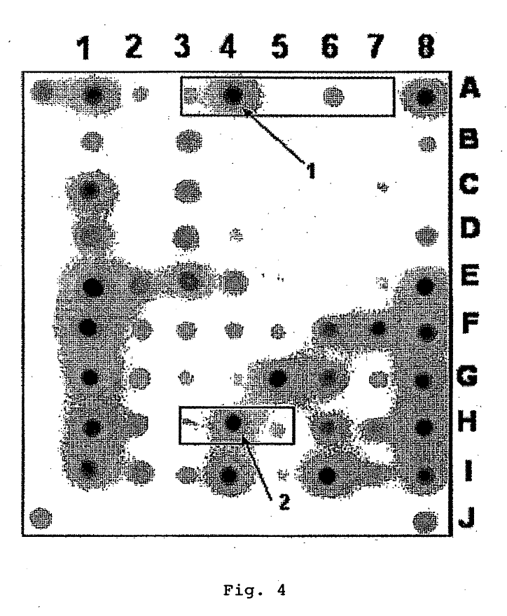

FIG. 4 shows the data of hybridization of DNA sample isolated from resistant to rifampicin and isoniazid strain of Mycobacterium tuberculosis complex.

In each group of biochip elements fluorescence intensity in the pad with oligonucleotide complimentary to wild type DNA is higher than the signal intensity in all other pads. Only groups of elements A3-A7 and H3-H5 are the exception (FIG. 4).

Element A4 demonstrates maximal fluorescence intensity within the group A3-A7. Therefore, DNA of the tested sample contains point nucleotide substitution C>G in the position 2384 of rpoB gene (Genbank Acc. No L27989) that leads to amino acid substitution Asp to Val in position No. 516 and results in the resistance of the tested strain to rifampicin.

Element H4 demonstrates maximal fluorescence intensity within group H3-H5. Therefore, the tested DNA contains point nucleotide substitution C>T in the position −15 relative to initiation translation site in promoter region of inhA gene.

Belonging of this strain to Mycobacterium tuberculosis complex is established by comparison of intensity of fluorescent signal in the pads with immobilized oligonucleotide complimentary to IS6110 (F8, I8) and in blank pads which play a role of negative control.

Therefore, it is concluded that Mycobacterium tuberculosis complex resistant to rifampicin and isoniazid is present in the tested clinical sample. Resistance to rifampicin is the result of amino acid substitution Asp to Val in the position No. 516 of rpoB gene. Resistance to isoniazid is the result of nucleotide substitution C>T (−15) in promoter region of inhA gene.

The presence of M. tuberculosis in sputum sample was confirmed by the cultivation on solid media and by biochemical tests. Therefore, the presence of drug-resistant M. tuberculosis was confirmed by the stable growth of colonies on the media with rifampicin and isoniazid that is in a whole agreement with the data obtained by the method of the present invention.

### Example 9

### Detection DNA Isolated from Strain of M. bovis BCG Sensitive to Rifampicin and Isoniazid by the Method of Hybridization on Biochip

Culture of characterized strain of M. bovis BCG belonging to Mycobacterium tuberculosis complex was analyzed by the method of the present invention as described in Examples 2-5.

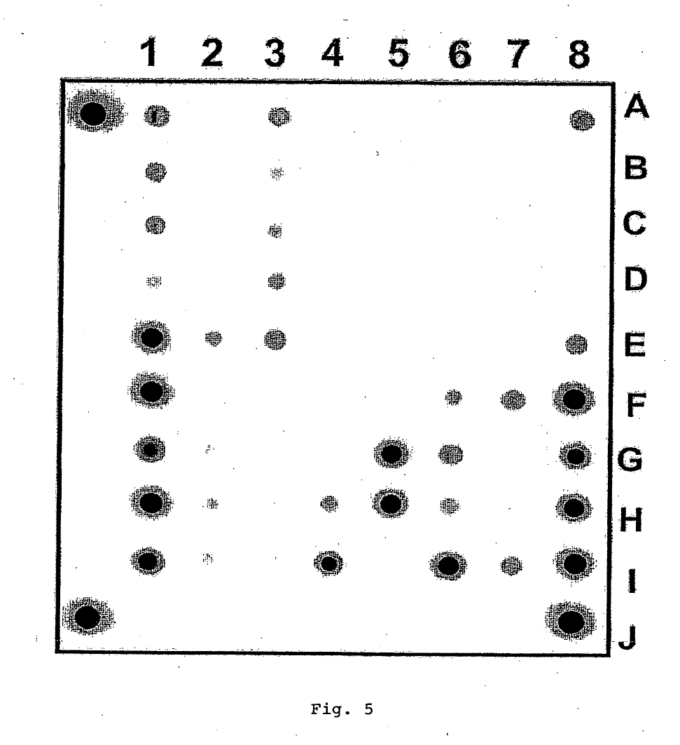

FIG. 5 shows the data of hybridization of DNA sample isolated from sensitive to rifampicin and isoniazid strain of Mycobacterium tuberculosis complex.

According to proposed algorithm of data interpretation the pads with oligonucleotides complimentary to wild type DNA have higher intensity of fluorescent signal in comparison with other pads inside each group. Therefore DNA of tested sample forms perfect hybridization duplexes with oligonucleotides complimentary to the sequence of wild type DNA (strain of Mycobacterium tuberculosis complex sensitive to rifampicin and isoniazid). Belonging of this strain to Mycobacterium tuberculosis complex is established by comparison of intensity of fluorescent signal in the pads with immobilized oligonucleotide complimentary to IS6110 (F8, I8) and in blank pads which play a role of negative control.

Therefore, it is concluded that analyzed DNA belongs to Mycobacterium tuberculosis complex sensitive to rifampicin and isoniazid. At the same time the hybridization pattern of DNA from strain M. bovis BCG coincides with hybridization pattern of wild type strain of M. tuberculosis.

### Example 10

### Analysis of DNA Isolated from Strain of M. avium S58 by the Method of Hybridization on Biochip

Culture of characterized strain of M. avium S58 which does not belong to Mycobacterium tuberculosis complex was analyzed by the method of the present invention as described in Examples 2-5.

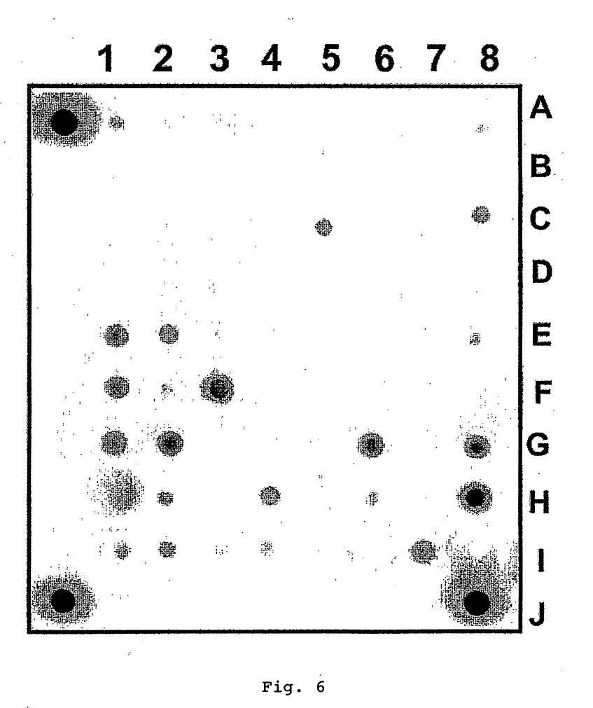

FIG. 6 shows the data of hybridization of DNA sample isolated from strain which does not belong to Mycobacterium tuberculosis complex.

According to proposed algorithm of data interpretation the belonging of analyzed strain to Mycobacterium tuberculosis complex is established by comparison of intensity of fluorescent signal in the pads with immobilized oligonucleotide complimentary to IS6110 (F8, I8) and in blank pads which play a role of negative control. As seen from FIG. 6, the signals in pads F8 and I8 on the hybridization pattern are absent. It means that genome of the tested microorganism does not contain insertion element IS6110. Therefore, analyzed DNA is isolated from microorganism which does not belong to Mycobacterium tuberculosis complex and an analysis of drug-resistance can be omitted.

Therefore, the present invention allows detect different forms of tuberculosis mycobacteria resistant to rifampicin and isoniazid rapidly with direct use of clinical material. Method of the present invention is favorably different from other currently existing analogs by simplicity of realization and low cost. Optimized two-staged multiplex PCR with the use of original set of primers allows to achieve high sensitivity (at least 500 genome-equivalents of target in the sample). The use of biochip containing original set of differentiating oligonucleotides allows also to conduct typing (marking) of tuberculosis mycobacteria on the level of genotype.

Although the preferred embodiments of the present invention and their advantages were described above in whole details the specialist in the art can easily add various changes and additions or, vice versa, omit something remaining in the scope of the present invention clarified below by the Claims.

1. Method for simultaneous detection of Mycobacterium tuberculosis complex and identification of the mutations in mycobacterial DNA, resulting in the resistance of microorganisms to rifampicin and isoniazid, in clinical samples which includes:
(A)—multiplex amplification of fragments of rpoB, katG, inhA, ahpC genes, IS6110 mobile element with the use of set of pairs of specific primers for the first PCR stage;
(B)—multiplex amplification of fragments of rpoB, katG, inhA, ahpC genes, IS6110 mobile element with the use of PCR product obtained on the stage (A) as template, and set of pairs of specific primers for the second PCR stage, where one of primers in each pair of primers is fluorescently labeled, to obtain predominantly single-strained fluorescently labeled fragments;
(C)—preparation of biochip for simultaneous detection of Mycobacterium tuberculosis complex and identification of mutations resulting in resistance to rifampicin and isoniazid, which consists of matrix with gel pads where in each of pads the unique oligonucleotide probe is immobilized where the sequence of oligonucleotide is chosen from the group including the sequences: a) corresponding to sequence of fragment of wild type rpoB gene; b) corresponding to sequence of fragment of mutant variant rpoB gene resulting in resistance of microorganisms to rifampicin; c) complementary to sequences described in a) and b); d) corresponding to sequence of fragment of wild type katG gene; e) corresponding to sequence of fragment of mutant variant katG gene resulting in resistance of microorganisms to isoniazid; complementary to sequences described in d) and e); g) corresponding to sequence of fragment of wild type inhA gene; h) corresponding to sequence of fragment of mutant variant inhA gene resulting in resistance of microorganisms to isoniazid; i) complementary to sequences described in g) and h); j) corresponding to sequence of fragment of wild type ahpC gene; k) corresponding to sequence of fragment of mutant variant ahpC gene resulting in resistance of microorganisms to isoniazid; l) complementary to sequences described in j) and k); m) corresponding to sequence of mobile element IS6110; o) complementary to sequence described in m).
(D)—hybridization of amplified labeled products obtained on stage (B) on biochip under conditions providing the one nucleotide resolution between matched and mismatched duplexes formed during hybridization;
(E)—registration and interpretation of the hybridization data.

2. Method as claimed in claim 1, characterized by the use on the first stage of multiplex PCR (A) of the set of pairs of specific primers the sequences of which are presented by SEQ ID NO: 70, 71, 74, 75, 77, 78, 80, 81, 83, 84.

3. Method as claimed in claim 1, characterized by the use on the second stage of multiplex PCR (B) of the set of pairs of specific primers the sequences of which are presented by SEQ ID NO: 72, 73, 74, 76, 77, 79, 80, 82, 83, 85.

4. Method as claimed in claim 1, characterized by the use on the second stage of multiplex PCR (B) of the fluorescently labeled primer in molar excess in relation to second primer from this pair to obtain predominantly single-strained fluorescently labeled fragments for all pairs of primers.

5. Method as claimed in claim 1, in which the amplification fragments of genes and mobile element IS6110 is carried out with the direct use of material from clinical sample (sputum, exudation, wash-out, bronchioalveolar lavage) or preliminary grown culture of microorganisms.

6. Method as claimed in claim 1, characterized by the use of biochip containing the set of immobilized oligonucleotides the sequences of which are presented by SEQ ID NO: 1-69.

7. Method as claimed in claim 1, characterized by the use of hybridization buffer which allows conducting of hybridization in expanded temperature interval to provide the one nucleotide resolution between matched and mismatched duplexes formed during hybridization.

8. Method as claimed in claim 1, characterized by the registration of the data on stage (E) with the use of portable fluorescence analyzer and software that allows to conduct automatic processing of signal intensities with following interpretation of the data.

9. Method as claimed in claim 1, where the interpretation of the data obtained on stage (E) is carried out by the comparison of fluorescence signal intensities in the pads in which matched and mismatched hybridization duplexes were formed.

10. Method as claimed in claim 1, where results of interpretation can be used for confirmation of clinical diagnosis of tuberculosis and for epidemiological genetic typing using presence of one or another mutation as a marker.

11. Set of specific pairs of primers for realization of the method for simultaneous detection of Mycobacterium tuberculosis complex and identification of mutations in mycobacterial DNA, resulting in the resistance of strains to rifampicin and isoniazid, in clinical samples where the sequences of primers are presented by SEQ ID NO: 70-85.

12. Biochip which is used in the method for simultaneous detection of Mycobacterium tuberculosis complex and identification of mutations in mycobacterial DNA, resulting in the resistance of strains to rifampicin and isoniazid, in clinical samples where the biochip consists of matrix with gel pads, where in each of pads the unique oligonucleotide probe is immobilized, and where the sequences of probes are presented by SEQ ID NO: 1-69.

13. Set of oligonucleotide probes which is used for preparation of biochip for the method of simultaneous detection of Mycobacterium tuberculosis complex and identification of mutations in mycobacterial DNA, resulting in the resistance of strains to rifampicin and isoniazid, in clinical samples, where the probes have the sequences SEQ ID NO: 1-69.


SEQUENCE LISTING (85 sequences)

SEQ ID NO: 1 - DNA, 16 bp, Mycobacterium tuberculosis
tggctggtgccgaaga

SEQ ID NO: 2 - DNA, 17 bp, Mycobacterium tuberculosis
ctcagctggctgaagaa

SEQ ID NO: 3 - DNA, 16 bp, Mycobacterium tuberculosis
ttggctcagctggctg

SEQ ID NO: 4 - DNA, 15 bp, Mycobacterium tuberculosis
ttggctcggctggct

SEQ ID NO: 5 - DNA, 15 bp, Mycobacterium tuberculosis
ttggctccgctggct

SEQ ID NO: 6 - DNA, 16 bp, Mycobacterium tuberculosis
aattggctcagctggc

SEQ ID NO: 7 - DNA, 15 bp, Mycobacterium tuberculosis
aattgggtcagctgg

SEQ ID NO: 8 - DNA, 14 bp, Mycobacterium tuberculosis
aattggcgcagctg

SEQ ID NO: 9 - DNA, 19 bp, Mycobacterium tuberculosis
tccatgaattggctcagct

SEQ ID NO: 10 - DNA, 17 bp, Mycobacterium tuberculosis
catgaataggctcagct

SEQ ID NO: 11 - DNA, 17 bp, Mycobacterium tuberculosis
catgaatttgctcagct

SEQ ID NO: 12 - DNA, 17 bp, Mycobacterium tuberculosis
catgaatgggctcagct

SEQ ID NO: 13 - DNA, 18 bp, Mycobacterium tuberculosis
ttctggtccatgaattgg

SEQ ID NO: 14 - DNA, 19 bp, Mycobacterium tuberculosis
gttctggtctatgaattgg

SEQ ID NO: 15 - DNA, 18 bp, Mycobacterium tuberculosis
ttgttctggtccatgaat

SEQ ID NO: 16 - DNA, 18 bp, Mycobacterium tuberculosis
ttgttctggaccatgaat

SEQ ID NO: 17 - DNA, 18 bp, Mycobacterium tuberculosis
ttgttctggtacatgaat

SEQ ID NO: 18 - DNA, 18 bp, Mycobacterium tuberculosis
ttgttctggcccatgaat

SEQ ID NO: 19 - DNA, 18 bp, Mycobacterium tuberculosis
ttgttctgctccatgaat

SEQ ID NO: 20 - DNA, 15 bp, Mycobacterium tuberculosis
tcaaccccgacagcg

SEQ ID NO: 21 - DNA, 15 bp, Mycobacterium tuberculosis
tcaaccccaacagcg

SEQ ID NO: 22 - DNA, 17 bp, Mycobacterium tuberculosis
ggcgcttgtgggtcaac

SEQ ID NO: 23 - DNA, 17 bp, Mycobacterium tuberculosis
ggcgcttgtcggtcaac

SEQ ID NO: 24 - DNA, 17 bp, Mycobacterium tuberculosis
ggcgcttgagggtcaac

SEQ ID NO: 25 - DNA, 17 bp, Mycobacterium tuberculosis
ggcgcttttgggtcaac

SEQ ID NO: 26 - DNA, 17 bp, Mycobacterium tuberculosis
ggcgcttgcaggtcaac

SEQ ID NO: 27 - DNA, 17 bp, Mycobacterium tuberculosis
ggcgcttgtaggtcaac

SEQ ID NO: 28 - DNA, 17 bp, Mycobacterium tuberculosis
ggcgcttgttggtcaac

SEQ ID NO: 29 - DNA, 17 bp, Mycobacterium tuberculosis
ggcgcttgcgggtcaac

SEQ ID NO: 30 - DNA, 17 bp, Mycobacterium tuberculosis
ggcgcttgggggtcaac

SEQ ID NO: 31 - DNA, 15 bp, Mycobacterium tuberculosis
cagcgccgacagtcg

SEQ ID NO: 32 - DNA, 16 bp, Mycobacterium tuberculosis
ccagcgccaacagtcg

SEQ ID NO: 33 - DNA, 15 bp, Mycobacterium tuberculosis
cagcgcccacagtcg

SEQ ID NO: 34 - DNA, 15 bp, Mycobacterium tuberculosis
cagcgcacacagtcg

SEQ ID NO: 35 - DNA, 15 bp, Mycobacterium tuberculosis
cagcgcctgcagtcg

SEQ ID NO: 36 - DNA, 17 bp, Mycobacterium tuberculosis
tgccccagcgccgacag

SEQ ID NO: 37 - DNA, 17 bp, Mycobacterium tuberculosis
tgccccggcgccgacag

SEQ ID NO: 38 - DNA, 16 bp, Mycobacterium tuberculosis
cgatcaccagcggcat

SEQ ID NO: 39 - DNA, 15 bp, Mycobacterium tuberculosis
cgatcaccacggcat

SEQ ID NO: 40 - DNA, 17 bp, Mycobacterium tuberculosis
cgatcaccacaggcatc

SEQ ID NO: 41 - DNA, 16 bp, Mycobacterium tuberculosis
gatcaccaacggcatc

SEQ ID NO: 42 - DNA, 16 bp, Mycobacterium tuberculosis
gatcaccatcggcatc

SEQ ID NO: 43 - DNA, 15 bp, Mycobacterium tuberculosis
gatcacccgcggcat

SEQ ID NO: 44 - DNA, 16 bp, Mycobacterium tuberculosis
atcaccagaggcatcg

SEQ ID NO: 45 - DNA, 15 bp, Mycobacterium tuberculosis
gatcaccggcggcat

SEQ ID NO: 46 - DNA, 15 bp, Mycobacterium tuberculosis
cgaaatgggacaaca

SEQ ID NO: 47 - DNA, 15 bp, Mycobacterium tuberculosis
cgaaaggggacaaca

SEQ ID NO: 48 - DNA, 16 bp, Mycobacterium tuberculosis
cgaaattggacaacag

SEQ ID NO: 49 - DNA, 15 bp, Mycobacterium tuberculosis
cgaaatgcgacaaca

SEQ ID NO: 50 - DNA, 18 bp, Mycobacterium tuberculosis
tcctcgagatcctgtacg

SEQ ID NO: 51 - DNA, 18 bp, Mycobacterium tuberculosis
tcctcgaggtcctgtacg

SEQ ID NO: 52 - DNA, 12 bp, Mycobacterium tuberculosis
ccggccgcggcg

SEQ ID NO: 53 - DNA, 12 bp, Mycobacterium tuberculosis
ccggcctcggcg

SEQ ID NO: 54 - DNA, 15 bp, Mycobacterium tuberculosis
gataggttgtcgggg

SEQ ID NO: 55 - DNA, 15 bp, Mycobacterium tuberculosis
gataggatgtcgggg

SEQ ID NO: 56 - DNA, 15 bp, Mycobacterium tuberculosis
gatagggtgtcgggg

SEQ ID NO: 57 - DNA, 15 bp, Mycobacterium tuberculosis
cggcgagacgatagg

SEQ ID NO: 58 - DNA, 15 bp, Mycobacterium tuberculosis
cggcgagatgatagg

SEQ ID NO: 59 - DNA, 14 bp, Mycobacterium tuberculosis
ggcgaggcgatagg

SEQ ID NO: 60 - DNA, 17 bp, Mycobacterium tuberculosis
gcacgatggaatgtcgc

SEQ ID NO: 61 - DNA, 18 bp, Mycobacterium tuberculosis
gcacgatagaatgtcgca

SEQ ID NO: 62 - DNA, 16 bp, Mycobacterium tuberculosis
tcacggcacgatggaa

SEQ ID NO: 63 - DNA, 16 bp, Mycobacterium tuberculosis
tcacggcatgatggaa

SEQ ID NO: 64 - DNA, 16 bp, Mycobacterium tuberculosis
tcacggcaagatggaa

SEQ ID NO: 65 - DNA, 16 bp, Mycobacterium tuberculosis
cacggcacgatggaat

SEQ ID NO: 66 - DNA, 16 bp, Mycobacterium tuberculosis
cacggcacaatggaat

SEQ ID NO: 67 - DNA, 16 bp, Mycobacterium tuberculosis
cttcacggcacgatgg

SEQ ID NO: 68 - DNA, 18 bp, Mycobacterium tuberculosis
acttcacggtacgatgga

SEQ ID NO: 69 - DNA, 17 bp, Mycobacterium tuberculosis
ccggagctgcgtgagcg

SEQ ID NO: 70 - DNA, 28 bp, Mycobacterium tuberculosis
cgtggaggcgatcacaccgcagacgttg

SEQ ID NO: 71 - DNA, 27 bp, Mycobacterium tuberculosis
agtgagacgggtgcacgtcgcggacct

SEQ ID NO: 72 - DNA, 20 bp, Mycobacterium tuberculosis
cgccgcgatcaaggagttct

SEQ ID NO: 73 - DNA, 20 bp, Mycobacterium tuberculosis
tcacgtgacagaccgccggg

SEQ ID NO: 74 - DNA, 27 bp, Mycobacterium tuberculosis
tggaagagctcgtatggcaccggaacc

SEQ ID NO: 75 - DNA, 24 bp, Mycobacterium tuberculosis
cggtgtattgccaagcgccagcag

SEQ ID NO: 76 - DNA, 27 bp, Mycobacterium tuberculosis
gctctccgtcagctcccactcgtagcc

SEQ ID NO: 77 - DNA, 24 bp, Mycobacterium tuberculosis
ccggaaatcgcagccacgttacgc

SEQ ID NO: 78 - DNA, 27 bp, Mycobacterium tuberculosis
ggtaaccaggactgaacgggatacgaa

SEQ ID NO: 79 - DNA, 23 bp, Mycobacterium tuberculosis
ggccccttcagtggctgtggcag

SEQ ID NO: 80 - DNA, 31 bp, Mycobacterium tuberculosis
atggtgtgatatatcacctttgcctgacagc

SEQ ID NO: 81 - DNA, 28 bp, Mycobacterium tuberculosis
ggaattgatcgccaatggttagcagtgg

SEQ ID NO: 82 - DNA, 27 bp, Mycobacterium tuberculosis
tgactctcctcatcatcaaagcggaca

SEQ ID NO: 83 - DNA, 30 bp, Mycobacterium tuberculosis
aggatggggtcatgtcaggtggttcatcga

SEQ ID NO: 84 - DNA, 30 bp, Mycobacterium tuberculosis
gcgaagaaagccgacgcggtctttaaaatc

SEQ ID NO: 85 - DNA, 22 bp, Mycobacterium tuberculosis
cgctgcccactccgaatcgtgc